SEQUENCE LISTING (20 sequences)

SEQ ID NO: 1 - DNA, 13 bp, Artificial sequence
  Single strand DNA oligonucleotide
gggggggggggga

SEQ ID NO: 2 - DNA, 13 bp, Artificial sequence
  Single strand DNA oligonucleotide
cccccccccccca

SEQ ID NO: 3 - DNA, 13 bp, Artificial sequence
  Single strand DNA oligonucleotide
agggggggggggg

SEQ ID NO: 4 - DNA, 13 bp, Artificial sequence
  Single strand DNA oligonucleotide
acccccccccccc

SEQ ID NO: 5 - DNA, 13 bp, Artificial sequence
  Single strand DNA oligonucleotide
ccccccccccccc

SEQ ID NO: 6 - DNA, 13 bp, Artificial sequence
  Single strand DNA oligonucleotide
ggggggggggggg

SEQ ID NO: 7 - DNA, 12 bp, Artificial sequence
  Single strand DNA oligonucleotide
gggggggggggg

SEQ ID NO: 8 - DNA, 13 bp, Artificial sequence
  Single strand DNA oligonucleotide
ccccccccccccc

SEQ ID NO: 9 - DNA, 12 bp, Artificial sequence
  Single strand DNA oligonucleotide
gggggggggggg

SEQ ID NO: 10 - DNA, 13 bp, Artificial sequence
  Single strand DNA oligonucleotide
ccccccccccccc

SEQ ID NO: 11 - DNA, 12 bp, Artificial sequence
  Single strand DNA oligonucleotide
gggggggggggg

SEQ ID NO: 12 - DNA, 13 bp, Artificial sequence
  Single strand DNA oligonucleotide
ccccccccccccc

SEQ ID NO: 13 - DNA, 12 bp, Artificial sequence
  Single strand DNA oligonucleotide
gggggggggggg

SEQ ID NO: 14 - DNA, 13 bp, Artificial sequence
  Single strand DNA oligonucleotide
ccccccccccccc

SEQ ID NO: 15 - DNA, 10 bp, Artificial sequence
  Single strand DNA oligonucleotide
gggggggggg

SEQ ID NO: 16 - DNA, 10 bp, Artificial sequence
  Single strand DNA oligonucleotide
cccccccccc

SEQ ID NO: 17 - DNA, 13 bp, Artificial sequence
  Single strand DNA oligonucleotide
ggtggggggggga

SEQ ID NO: 18 - DNA, 13 bp, Artificial sequence
  Single strand DNA oligonucleotide
gggtgggggggga

SEQ ID NO: 19 - DNA, 13 bp, Artificial sequence
  Single strand DNA oligonucleotide
ggggtggggggga

SEQ ID NO: 20 - DNA, 13 bp, Artificial sequence
  Single strand DNA oligonucleotide
gggggtgggggga

(19) United States
(12) Patent Application Publication
(10) Pub. No.: US 2006/0134680 A1
(43) Pub. Date: Jun. 22, 2006

(54) Homogeneous populations of nucleic acids, methods of synthesyzing same and uses thereof

(76) Inventors: Alexander Kotlyar, Zoran (IL); Natalia Borovok, Ariel (IL); Danny Porath, Jerusalem (IL)

Correspondence Address: Martin D. MOYNIHAN;PRTSI, Inc., P.O. Box 16446, Arlington, VA, 22215, US

(21) Appl. No.: 11/312,385

(22) Filed: Dec. 21, 2005

Related U.S. Application Data
(60) Provisional application No. 60/637,743, filed on Dec. 22, 2004.

(51) Int. Cl. C12Q 1/68; C07H 21/02; C12P 19/34; B32B 27/04; C12M 1/34; C07H 21/04

(52) U.S. Cl. 435/6; 435/91.2; 435/287.1; 536/23.1; 977/706; 442/128

(57) ABSTRACT

A homogeneous population of fully double stranded nucleic acid molecules having blunt ends, each of the molecules being at least 100 base pairs long, and having the same repetitive core sequence selected from the group consisting of mono, di-, or tri-nucleotide, with the proviso that the repetitive core sequence is not A mononucleotide is disclosed. Methods of synthesis and uses thereof are also disclosed.

### RELATED APPLICATIONS

This application claims the benefit of priority from U.S. Provisional Patent Application No. 60/637,743, filed on Dec. 22, 2004, the contents of which are incorporated herein by reference.

### FIELD AND BACKGROUND OF THE INVENTION

The present invention relates to homogeneous populations of nucleic acids and methods of synthesizing same. More particularly, the present invention relates to a homogeneous population of poly(dG)-poly(dC) and its applications in nanoelectronics.

Nanoscience is the science of small particles of materials and is one of the most important research frontiers in modem technology. These small particles are of interest from a fundamental point of view since they enable construction of materials and structures of well-defined properties. With the ability to precisely control material properties arise new opportunities for technological and commercial development, and applications of nanoparticles have been shown or proposed in areas as diverse as micro- and nanoelectronics, nanofluidics, coatings and paints and biotechnology.

It is well established that future development of microelectronics, magnetic recording devices and chemical sensors will be achieved by increasing the packing density of device components. Traditionally, microscopic devices have been formed from larger objects, but as these products get smaller, below the micron level, this process becomes increasingly difficult. It is therefore appreciated that the opposite approach is to be employed, essentially, the building of microscopic devices from a molecular level up, primarily via objects of nanometric dimensions.

In particular, wire-like conducting or semiconducting nanostructures have attracted extensive interest over the past decade due to their great potential for addressing some basic issues about dimensionality and space confined transport phenomena as well as related applications. The DNA molecule, well known from biology for containing the genetic code of all living species, has recently caught the attention of chemists and physicists as a possible candidate to wire electronic materials in a programmable way by virtue of its recognition and self-assembling properties [Di Mauro E, Hollenberg C P (1993) Adv Mat 5:384].

For DNA to be relevant as a molecular wire it must both conduct a current and have the capability of attachment to surfaces. Several methods have successfully been used to attach DNA to a surface. For example, a DNA molecule can be functionalized with a thiol (S—H) or a disulfide (S—S) group at the 3′ or 5′ end and thus bind to metals such as gold and platinum. DNA has also been covalently bound to preactivated particle surfaces. In addition, incorporation of biotin in the DNA molecule allows the DNA to bind to particle surfaces coated with avidin.

There is currently a heated debate whether native DNA can mediate long range electron transfer. For a long time DNA was thought of as an insulator and only recently have some experiments suggested that this might not be true. Various works have suggested that DNA is poorly conductive [P. Tran, et al., Phys. Rev. Lett. 85 (2000) 1564], whereas some suggest that DNA possesses highly conductive properties [H. W. Fink, C. SchVonenberger, Nature 398 (1999) 407]. According to this view a double helical DNA molecule can be treated as a π stacked conductivity system which allows electrons to move effortlessly as a current through an electrical wire.

It has been shown that efficiency of charge transfer is reduced in nucleic acid duplexes containing mismatches and bulges. Proteins that bind and disrupt continuous base-stacking in duplex DNA also reduce the efficiency of electron transfer past the site of helix disruption. It has also been demonstrated that uniform DNA comprising repeating sequences improves conduction properties. Thus, DNA composed of repeating sequences has been shown to conduct better than DNA composed of random sequences [Hennig, D., et al., (2004). J. Biol. Phys., 30, 227-238].

Specifically, it is thought that poly(dG)-poly(dC) provides the best conditions for π overlap. In addition, guanines which have the lowest ionization potential among DNA bases, promote charge migration through the DNA. Recent experimental demonstration of the conducting behavior in short poly(dG)-poly(dC) DNA oligomers [Porath, D., et al., (2000) Nature, 403, 635-638] and the results of theoretical calculations showing that poly(dG)-poly(dC) exhibits better conductance than poly(dA)-poly(dT), [Hennig, D., et al., (2004). J. Biol. Phys., 30, 227-238] support an idea of possible application of poly(dG)-poly(dC) in molecular electronic devices.

Commercial preparations of poly(dG)-poly(dC) are manufactured by Amersham Biosciences Company (Sweden) in which the DNA polymer is synthesized using the Klenow fragment of DNA Polymerase 1, dGTP, dCTP, and Poly(dI)-Poly(dC) as the template-primer. These preparations used by researchers in electrical conductivity studies [Yoo, K. H. et al., (2001) Phys. Rev. Lett, 87, 198102], however, have a number of disadvantages. They are characterized by a broad size distribution of the molecules and the presence of single stranded fragments along the DNA. The presence of irregular fragments and strand breaks along poly(dG)-poly(dC) may strongly reduce the ability of the wires to conduct a current.

There is thus a widely recognized need for, and it would be highly advantageous to generate homogeneous populations of double stranded DNA devoid of the above limitations.

### SUMMARY OF THE INVENTION

According to one aspect of the present invention there is provided a homogeneous population of fully double stranded nucleic acid molecules having blunt ends, each of the molecules in the population being at least 100 base pairs long, each of the molecules in the population having the same repetitive core sequence selected from the group consisting of mono-, di-, or tri-nucleotide, with the proviso that the repetitive core sequence is not A mononucleotide.

According to another aspect of the present invention there is provided a method of synthesizing a homogeneous population of fully double stranded nucleic acid molecules having blunt ends, the method comprising reacting a fully double stranded initiator nucleic acid molecule having blunt ends and further having a repetitive core sequence selected from the group consisting of mono-, di-, or tri-nucleotide with nucleotide tri phosphates in the presence of a nucleic acid polymerase, thereby de novo enzymatically synthesizing the homogeneous population of fully double stranded nucleic acid molecules having blunt ends.

According to yet another aspect of the present invention there is provided a kit for de novo synthesizing a homogeneous population of nucleic acid molecules, the kit comprising, in a single container nucleotides and fully double stranded initiator nucleic acid molecules, the fully double stranded initiator nucleic acid molecules having blunt ends and further having a repetitive core sequence selected from the group consisting of mono-, di-, or tri-nucleotide.

According to still another aspect of the present invention there is provided a wire composed of the fully double stranded nucleic acid molecules having blunt ends.

According to an additional aspect of the present invention there is provided a fiber composed of the fully double stranded nucleic acid molecules having blunt ends.

According to yet an additional aspect of the present invention there is provided a fabric composed of the fully double stranded nucleic acid molecules having blunt ends.

According to still an additional aspect of the present invention there is provided an electronic circuit comprising a support and fully double stranded nucleic acid molecules having blunt ends connected to each other and attached to the support.

According to a further aspect of the present invention there is provided a homogeneous population of fully double stranded nucleic acid molecules having blunt ends, each of the molecules in the population being at least 1000 base pairs long, each of the molecules in the population having the same repetitive core sequence of an A mononucleotide.

According to yet a further aspect of the present invention there is provided a composition comprising the homogeneous population of fully double stranded nucleic acid molecules having blunt ends.

According to further features in preferred embodiments of the invention described below, each fully double stranded nucleic acid molecule is less than ten kilobases.

According to still further features in the described preferred embodiments the mononucleotide comprises guanine.

According to still further features in the described preferred embodiments a 5′ end of at least one of the fully double stranded nucleic acid molecules is attached to a functional moiety.

According to still further features in the described preferred embodiments the functional moiety is selected from the group consisting of a thiol molecule, a disulfide molecule and a biotinylated molecule.

According to still further features in the described preferred embodiments the mono-, di, or trinucleotide comprises at least one modified base.

According to still further features in the described preferred embodiments at least one of the fully double stranded nucleic acid molecules is immobilized to a solid support.

According to still further features in the described preferred embodiments at least one of the fully double stranded nucleic acid molecules comprises a material selected from the group consisting of a conducting material, a semiconducting material, a thermoelectric material, a magnetic material, a light-emitting material, a biomineral and a polymer.

According to still further features in the described preferred embodiments the conducting material is a transition metal.

According to still further features in the described preferred embodiments the transition metal is selected from the group consisting of silver, gold, copper, platinum, nickel and palladium.

According to still further features in the described preferred embodiments the semiconducting material is selected from the group consisting of a group IV semiconducting material, a group II-VI semiconducting material and a group III-V semiconducting material.

According to still further features in the described preferred embodiments the magnetic material is a paramagnetic material.

According to still further features in the described preferred embodiments the paramagnetic material is selected from the group consisting of aluminum, copper, and platinum.

According to still further features in the described preferred embodiments the magnetic material is a ferromagnetic material.

According to still further features in the described preferred embodiments the ferromagnetic material is selected from the group consisting of magnetite, cobalt, nickel and iron.

According to still further features in the described preferred embodiments the light-emitting material is selected from the group consisting of dysprosium, europium, terbium, ruthenium, thulium, neodymium, erbium, ytterbium and any organic complex thereof.

According to still further features in the described preferred embodiments the biomineral comprises calcium carbonate.

According to still further features in the described preferred embodiments the polymer is selected from the group consisting of polyethylene, polystyrene and polyvinyl chloride.

According to still further features in the described preferred embodiments the thermoelectric material is selected from the group consisting of bismuth telluride, bismuth selenide, bismuth antimony telluride and bismuth selenium telluride.

According to still further features in the described preferred embodiments the functional moiety is a detectable moiety.

According to still further features in the described preferred embodiments the fully double stranded initiator nucleic acid molecule are at least ten nucleotides long.

According to still further features in the described preferred embodiments the fully double stranded initiator nucleic acid molecule is purified.

According to still further features in the described preferred embodiments the mono nucleotide comprises guanine.

According to still further features in the described preferred embodiments the fully double stranded initiator nucleic acid molecule is between ten and twenty.

According to still further features in the described preferred embodiments the reacting is effected at a non denaturing temperature.

According to still further features in the described preferred embodiments the non-denaturing temperature is between 25° C. and 37° C.

According to still further features in the described preferred embodiments the nucleic acid polymerase is exonuclease free Klenow.

According to still further features in the described preferred embodiments the method further comprises isolating the homogeneous population of nucleic acid molecules following the reacting.

According to still further features in the described preferred embodiments the kit further comprises polymerase in a separate container.

The present invention successfully addresses the shortcomings of the presently known configurations by providing a method for producing homogenous populations of nucleic acid molecules having a repeating sequence of no more than three nucleotides, and being over one hundred base pairs long.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a photograph of poly(dG)-poly(dC) molecules stained with ethidium bromide and electrophoresed on a 1% agarose gel. Molecular weights of 1 Kb DNA-ladder (lane 1) are indicated by left side arrows; poly(dG)-poly(dC) from Sigma, St. Louis, lot 103K10561 (lane 2); poly(dG)-poly(dC) from Sigma treated for 30 minutes at 70° C. (lane 3); poly(dG)-poly(dC) synthesized as described in Example 1 using HPLC-purified 0.2 μM (dG)₁₀-(dC)₁₀ as template-primer and 40 μg/ml of Klenow exo- (lane 4); poly(dG)-poly(dC) synthesized as described in Example 1 using 0.2 μM (dG)₁₀-(dC)₁₀ not specially purified by HPLC as template-primer and 40 μg/ml of Klenow exo- (lane 5). The electrophoresis was conducted for 1 hour at 130 V. The amount of DNA loaded per lane was approximately 20 ng;

FIGS. 2A-C are graphs illustrating the HPLC elution profile of poly(dG)-poly(dC) at high pH. FIG. 2A illustrates the HPLC elution profile of Poly(dG)-poly(dC) synthesized with Klenow exo- as described in FIG. 1 (solid curve) and Poly(dG)-Poly(dC) from Sigma, (dashed curve) were pretreated for 15 minutes at room temperature in 0.1 M KOH. 100 μl of each DNA sample were applied onto TSKgel G-DNA-PW column (7.8×300 mm) and eluted at room temperature with 0.1 M KOH at a flow rate of 0.5 ml/min. Elution was followed at 260 nm. FIGS. 2B and 2C illustrate normalized absorbance spectra for commercial Poly(dG)-Poly(dC) (Sigma) obtained using diode-array detection of fractions eluted at the time points indicated by the arrows;

FIGS. 3A-B are graphs illustrating the HPLC separation of products of Poly(dG)-Poly(dC) synthesis. FIG. 3A illustrates the size-dependent HPLC separation of the products of Poly(dG)-Poly(dC) synthesis. Polymerase extension assay was performed as described in Example 1 with 0.2 μM (dG)₁₀-(dC)₁₀ and 20 μg/ml Klenow exo- at 37° C. Polymerization reaction was started by addition of the enzyme. 50 μl of aliquots were withdrawn from the assay mixture before (black curve) and 30 (red curve), 60 (green curve) and 120 (blue curve) minutes after the addition of the enzyme and loaded on TSKgel G-DNA-PW column (7.8×300 mm). Elution was performed with 20 mM Tris-Acetate buffer, pH 7.0, at a flow rate of 0.5 m/min. FIG. 3B illustrates the anion-exchange HPLC separation of nucleotides. Nucleotide peaks from corresponding size-exclusion separation (FIG. 3A) were collected and loaded on an anion-exchange PolyWAX LP column (4.6×200 mm). Elution was performed using a 30 min linear K-Pi, pH 7.4, gradient between 0.02 and 0.5 M in the presence of 10% Acetonitrile at a flow rate of 0.8 ml/min. Elution was followed at 260 nm;

FIG. 4 is a CD spectrum of 3 Kbp poly(dG)-poly(dC) (30 nM per molecules) recorded in 20 mM Tris-Acetate buffer, pH 7.0, at 25° C. on Aviv Model 202 series (Aviv Instrument Inc., USA) Circular Dichroism Spectrometer. The spectrum was recorded from 220 to 320 nm and was an average of five scans. Recording specifications were: wavelength step 0.5 nm, settling time 0.333 seconds, average time 1.0 second, bandwidth 1.0 nm, path length 1 cm;

FIGS. 5A-B illustrate the time course of poly(dG)-poly(dC) synthesis. Polymerase extension assay was performed as described in Example 1 with 0.2 μM (dG)₁₀-(dC)₁₀ and 20 μg/ml Kienow exo-; the incubation was at 37° C. Aliquots were withdrawn every 15 minutes for 2 hours and 15 minutes. FIG. 5A is a photograph of the reaction products resolved on a 1% agarose gel and stained with ethidium bromide under conditions described in Example 1. The marker bands of 1 Kb DNA Ladder (lane 1) are indicated on the left. Time dependent products for 15, 30, 45, 60, 75, 90, 105, 120, and 135 min of the synthesis were run in lanes 2-10. FIG. 5B is a plot graph illustrating the dependence of polymer length (in Kbase pairs) estimated from FIG. 5A on the time of synthesis;

FIGS. 6A-C are graphs illustrating FRET in Fluorescein-(dG)₁₂-(dC)₁₂-TAMRA (tetramethylrhodamine) during extension by Klenow exo-. FIG. 6A is a time course of Fluorescein (Flu) emission. Polymerase extension assay was performed as described in Example 1 with 5 μM Flu-(dG)₁₂-(dC)₁₂-TAMRA and 0.8 μg/ml Kienow exo-. The assay mixture containing Flu-(dG)₁₂-(dC)₁₂-TAMRA and nucleotides was transferred into a fluorimetric cuvette. Fluorescence emission at 520 nm was recorded against time as described in Example 2; excitation was at 490 nm. A significant amount of energy transfer was detected as a large decrease in the contribution of the Flu donor and an increase in the contribution of the TAMRA acceptor. The extension reaction was started by addition of the enzyme and fluorescence was recorded in time. FIG. 6B is a plot of absorbance spectra of the synthesized polymer at increasing wavelengths. 0.5 ml of sample aliquots was withdrawn from the incubation prior to (curve 1) and 5 (curve 2), 10 (curve 3), 20 (curve 4), 30 (curve 5), and 40 (curve 6) minutes following addition of the enzyme to the assay. The samples were passed through Sephadex G-25 DNA Grade column (1×5 cm) in 20 mM Tris-Acetate buffer, pH 8.0, to separate high molecular weight products of the synthesis from nucleotides; absorption spectra of the synthesized polymer eluted in the column's void volume were recorded. FIG. 6C is a plot of polymer length against time. The amount of G-C base pairs in double-labeled product of the synthesis were estimated from analysis of the spectra presented in FIG. 6B as described in Example 2, and plotted as a function of time of synthesis;

FIG. 7 is a plot of Fluorescein emission spectra of the products of Flu-(dG)₁₂-(dC)₁₂-TAMRA extension. Polymerase extension assay was performed as described for FIG. 6. The spectra were recorded prior to (curve 1), and 10 (curve 2) and 25 (curve 3) minutes following initiation of the synthesis. Excitation was at 490 nm. Schematic presentations of corresponding double-stranded products of the synthesis are indicated to the right; F denotes Flu, T denotes TAMRA. A significant amount of energy transfer in Flu-(dG)₁₂-(dC)₁₂-TAMRA is apparent as a decrease in the contribution of the Flu donor and an increase in the contribution of the TAMRA acceptor. The latter is seen as an increased relative emission around 580 nm in spectra of Flu-(dG)₁₂-(dC)₁₂-TAMRA. Extension of Flu-(dG)₁₂-(dC)₁₂-TAMRA results in increase of molecular distance between the dyes and, as a result, in increase of Flu emission. When the length of extended polymer reaches approximately 20 base pairs, a reduced amount of energy transfer is apparent (curve 2). Flu emission reaches maximum, when the length of extended polymer is equal to approximately 30 base pairs (˜10 nm); no contribution of TAMRA emission is then seen;

FIG. 8 is an absorbance profile as measured by HPLC analysis of products of early phase of poly(dG)-poly(dC) synthesis. Polymerase extension assay was performed as described in Example 1, with 15 μM (dG)₁₀-(dC)₁₀ and 2 μg/ml Klenow exo- at 37° C. The reaction was started by addition of the enzyme and was terminated by addition of 10 mM EDTA. 50 μl aliquots were withdrawn from the assay mixture prior to (continuous curve) and 5 minutes (dashed curve) following the start of the reaction. Oligonucleotides were separated from dGTP and dCTP with TSKgel G-3000 SWXL HPLC column (7.8×300 mm) and loaded in 0.1M KOH onto TSKgel DNA-NPR column (4.6×75 mm) equilibrated with 0.1M KOH. Elution was performed using a 30 minute linear KCl gradient between 0 and 1 M in 0.1 M KOH at a flow rate of 0.6 ml/min. Elution of correspondent C and G-strands are indicated in the Figure;

FIG. 9 is an absorbance profile as measured by HPLC analysis of products of 5′CCCCCCCCCCCCA3′ (SEQ ID NO: 2) and 5′GGGGTGGGGGGGA3′ (SEQ ID NO: 19) extension. Polymerase extension assay was performed as described in Example 1 with 5 μM 5′CCCCCCCCCCCCA3′ and 5′GGGGTGGGGGGGA3′ and 10 μg/ml Klenow exo- at 37° C. The reaction was started by addition of the enzyme and terminated by addition of 10 mM EDTA. 50 μl sample aliquots were withdrawn from the assay mixture prior to (black curve) and 10 (red curve), and 20 (blue curve) minutes after the reaction had been started. Oligomers were separated from dGTP and dCTP with TSKgel G-3000 SWXL HPLC column (7.8×300 mm) and loaded in 0.1 M KOH onto TSKgel DNA-NPR column (4.6×75 mm). Elution was performed using a 20 minute linear gradient between 0 and 1 M KCl in 0.1 M KOH at a flow rate of 0.6 ml/min; and

FIG. 10 is a model for 5′CCCCCCCCCCCCA3′ (SEQ ID NO: 2) and 5′GGGGTGGGGGGGA3′ (SEQ ID NO: 19) extension. The figure depicts the assumed events during extension of double stranded 5′CCCCCCCCCCCCA3′-5′GGGGTGGGGGGGA3′ oligonucleotide. Polymerase binds the oligonucleotide (1) and shifts A nucleotide at the 3′-end of 5′CCCCCCCCCCCCA3′ until it is becoming paired with T. A single stranded template-primer fragment and a loop de novo are then formed as a result of the 3′-end slippage (2). The primer strand is synthesized complementary to the template sequence; residues incorporated into primer are marked in red (3). The enzyme-DNA complex dissociates (4) and a loop relaxes into a structure with overhang at the 5′-end (5). The overhang cannot be used as a template for Klenow exo- due to inability to pair A-nucleotide at the 3′-end of the primer with either nucleotide in sequence of the template.

### DESCRIPTION OF THE PREFERRED EMBODIMENTS

The present invention is of homogeneous populations of nucleic acids and methods of production thereof.

The nucleic acid molecules of the present invention can be used as wire-like conducting or semiconducting nanostructures in the field of nanoelectronics.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Charge migration along DNA molecules has been the subject of scientific interest for over half a century. There is significant evidence to suggest that native DNA may comprise conducting abilities which has, in turn, given rise to a search for the DNA molecule with a structure and sequence that would best enable the conducting of a current.

Poly(dG)-poly(dC) is a potential candidate as it provides the best conditions for π overlap. Commercial preparations of poly(dG)-poly(dC) however, have a number of disadvantages. They are characterized by a broad size distribution of the molecules and the presence of single stranded fragments along the DNA. The presence of irregular fragments and strand breaks along poly(dG)-poly(dC) may strongly reduce the ability of the wires to conduct a current.

While reducing the present invention to practice, the present inventors have devised a novel method for synthesizing homogeneous populations of fully double stranded nucleic acid molecules, such as poly(dG)-poly(dC).

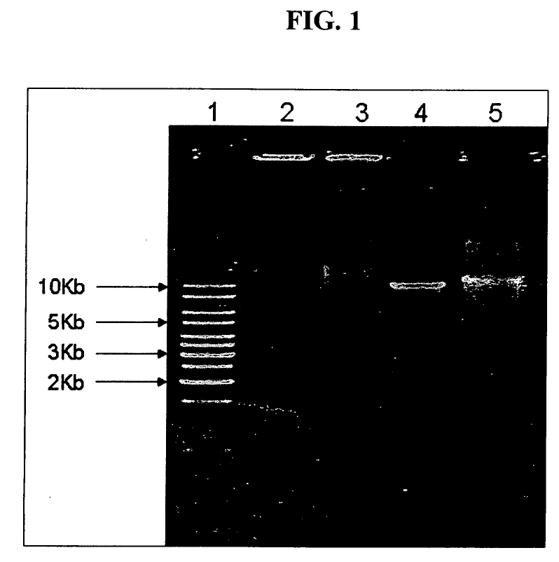

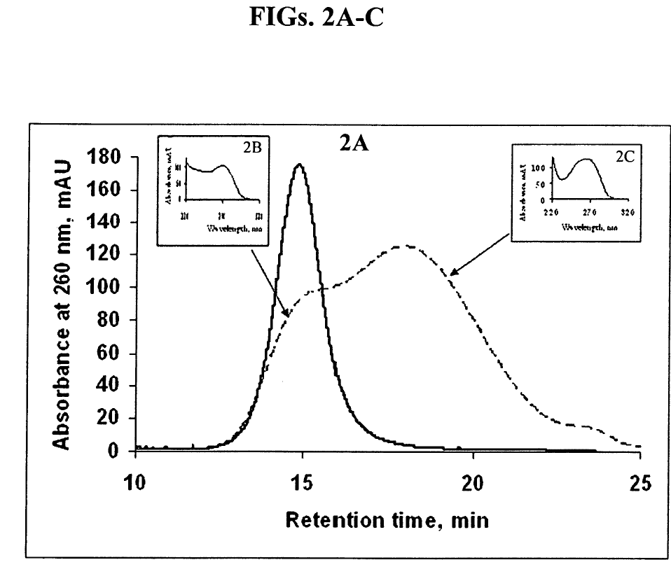

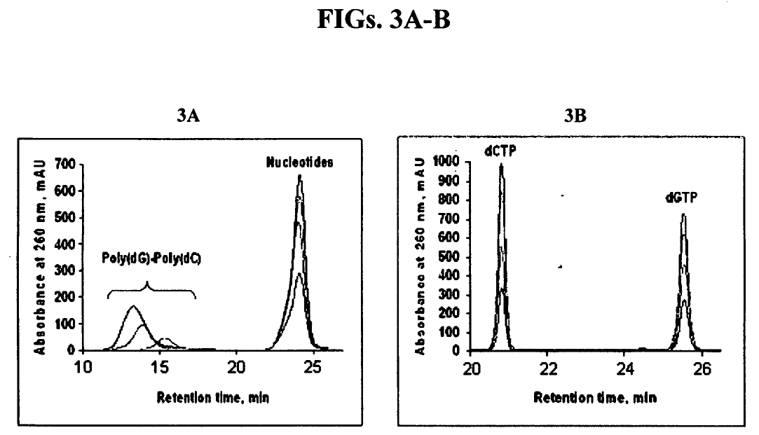

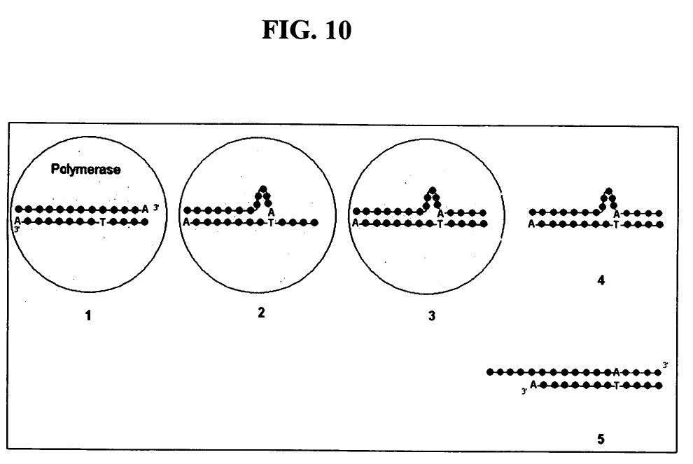

As is illustrated hereinbelow and in the Examples section which follows the present inventors have uncovered that incubation of a purified poly(dG) oligodeoxynucleotide with a purified poly(dC) oligodeoxynucleotide of the same length under conditions where the two hybridize to produce a fully double stranded molecule, together with polymerase and nucleotides generates a homogeneous population of fully double stranded molecules up to 10 kb in length. A model for the mechanism of synthesis is provided in FIG. 10. The nucleic acid molecules of the present invention were shown by gel electrophoresis to be uniform in length (FIG. 1) and to comprise equal quantities of guanine base and cytosine base as measured by the HPLC elution profile of poly(dG)-poly(dC) at high pH (FIG. 2A) and by anion-exchange HPLC separation of nucleotides following poly(dG)-poly(dC) synthesis (FIGS. 3A-B).

It should be noted that in sharp contrast to the present invention, prior art compositions such as those described by Schachman, et al and Radding et al were heterogeneous since initiator molecules which were not fully double stranded were used [Schachman et al., 1960, J. Biol. Chem., 235, 3242-3249; Radding, C. M., (1962) J. Biol. Chem., 237, 2869-2876]. As mentioned hereinabove, the present inventors proved that the use of fully double stranded initiator molecules is critical for the uniformity of length of the synthesized product. Radding et al teach de novo synthesis of poly(dG)-poly(dC) initiator molecules using the same reactants as those described by Schachman for poly(dAdT)—(i.e. polymerase, MgCl₂ and corresponding nucleotides). Analysis of the initiator molecules by Radding revealed that they were not fully double stranded as they comprised unequal amounts of dGTP and dCTP.

Tanaka et al teach synthesis of poly(dG)-poly(dC) up to 500 base pairs long using initiator molecules which are not fully double stranded [Tanaka et al., Chem Commun (Camb). 2004 Nov. 7; (21):2388-9]. As shown by Tanaka et al, the synthesized product was not homogeneous as it had a wide size distribution following electrophoresis.

Using a similar method, Tanaka et al synthesized poly(dA)-poly(dT) with non-fully double stranded initiator molecules [Tanaka et al., Chem Commun (Camb). 2002 Oct. 21; (20):2330-l]. This yielded compositions of fully double stranded poly(dA)-poly(dT) molecules 1000 base pairs long contaminated with 2000 base pair long molecules. The method of the present inventors yields a non-contaminated homogeneous population of molecules up to 10 kilobase pairs long.

Accordingly the compositions of the present invention are superior over those described by each Schachman et al, Radding et al and Tanaka et al.

Thus, according to one aspect of the present invention, there is provided a method of synthesizing a homogeneous population of fully double stranded nucleic acid molecules having blunt ends, the method comprising reacting a purified fully double stranded initiator nucleic acid molecule having blunt ends and further having a repetitive core sequence selected from the group consisting of mono-, di-, or tri-nucleotide with nucleotide tri phosphates in the presence of a nucleic acid polymerase, thereby de novo enzymatically synthesizing the population of fully double stranded nucleic acid molecules having blunt ends.

As used herein, the term “synthesizing” refers to a process of enzymatic polymerizing. According to this aspect of the present invention, the synthesizing is de novo.

As used herein, the phrase “enzymatically synthesizing” refers to synthesis due to the activity of a nucleic acid polymerase that incorporates nucleotide triphosphates onto the 3′ hydroxyl terminus of a nucleic acid in a 5′ to 3′ direction.

As used herein, the term “de novo” refers to a synthesis wherein the fully double stranded nucleic acid molecule end-product is longer than the fully double stranded initiator nucleic acid molecule reactant.

As used herein, the term “reacting” refers to the bringing together of chemical (e.g., biological) reagents in such a manner so as to allow their interaction at the molecular level to achieve a chemical or physical transformation.

As used herein, the term “homogeneous population” refers to a population of nucleic acid molecules whose members have an identical nucleotide composition across a substantially uniform length.

The phrase “substantially uniform length” refers to a difference in length that is no more than 10% of the average length of the homogeneous population of fully double stranded nucleic acid molecules. The homogeneous population of nucleic acid molecules of the present invention is further described hereinbelow.

As used herein, the phrase “fully double stranded nucleic acid molecules having blunt ends” refers to a nucleic acid molecule which is in full Watson-Crick base pairing. Thus, both the homogeneous population of nucleic acid molecules and the initiator nucleic acid molecules of the present invention comprise no 5′ overhangs or other single-stranded elements.

As used herein the term “purified” refers to removal of at least 80% and even more preferably 90% of the contaminating molecules that participate in the synthesis process and/or are a by-product of the synthesis process. As can be seen from the Examples section below, single stranded oligodeoxynucleotide molecules which have not undergone purification do not initiate the synthesis of a homogeneous population of product nucleic acid molecules.

Methods of purifying single stranded oligodeoxynucleotide molecules according to the teachings of the present invention include, but are not limited to ion exchange chromatography or HPLC. Thus, as demonstrated in Example 1 below, poly(dC)-single stranded oligodeoxynucleotide molecules were purified using an ion-exchange Western Analytical Products (USA) PolyWax LP column (4.6×200 mm, 5 μm, 300 Å) at pH 7.5. Poly(dG)-single stranded oligodeoxynucleotide molecules were purified using a ion-exchange HiTrap QHP column (5×1 ml) from Amersham-Biosciences (Sweden) in 0.1M KOH. Modified single stranded oligodeoxynucleotide molecules were purified by HPLC using a Vydac (USA) reverse-phase C₁₈ column (4.6×250 mm). HPLC single stranded oligodeoxynucleotide molecules were then desalted using pre-packed Sephadex G-25 DNA-Grade columns (Amersham-Biosciences, Sweden).

As used herein the term “nucleotide triphosphates” refers to any nucleotide triphosphate (NTP) which may be incorporated into a nucleic acid by DNA polymerase that does not prevent the synthesis of the homogeneous population of nucleic acid molecules of the present invention. Examples of nucleotide triphosphates include deoxynucleotide triphosphates such as adenosine triphosphates, guanosine triphosphates, cytidine triphosphates and thymidine triphosphates. Examples of other deoxynucleotide triphosphates which may be incorporated into the nucleic acid molecules of the present invention include deoxyuridine triphosphates [Yoshida S. Biochim. Biophys. Acta. 1979 Feb. 27; 561(2):396-402] and those with modified bases such as deoxyinosine triphosphates [Ji Hyung Chung et al., Nucleic Acids Research, 2001, Vol. 29, No. 14 3099-3107]. Other examples of deoxynucleotide triphosphates comprising modified bases are described herein below. According to the teachings of the present invention, two deoxynucleotide triphosphates which base pair to the same complementary nucleotide triphosphate may not be added in the same reaction, otherwise the product may not be homogeneous. Thus, for example, a mixture of uridine triphosphate and thymidine triphosphate may not be added in a single reaction. Similarly, a modified dNTP may not be included in the reaction unless it is capable of 100% incorporation into the homogeneous population of nucleic acid molecules.

It will be appreciated that the nucleotides present in the reaction mixture must include nucleotides which are complementary to the initiator molecule, although other nucleotides may still be present.

The initiator nucleic acid molecules of the present invention serve as both primers and templates for the synthesis reaction (see FIG. 10). According to this aspect of the present invention, the initiator nucleic acid molecules are fully double stranded and have a repetitive core sequence selected from the group consisting of mono-, di-, or tri-nucleotide. Without being bound to theory, it is believed that the polymerase enzyme binds to the 3′-end of the initiator nucleic acid molecule, shifts the end-nucleotide on the 3′-end of the initiator nucleic acid molecule in 5′-direction until it base pairs with the next complementary nucleotide and generates a short, single-stranded template and a loop de novo. Loop migration through the initiator nucleic acid molecule results in formation of a template region on its opposite end; filling the template by polymerase finalizes a single extension cycle.

Typically, the initiator nucleic acid molecules of the present invention comprise natural or synthetic nucleotides which are capable of base pairing (e.g. deoxynucleotide). Examples include the purine derivatives deoxyadenosine or deoxyguanosine and the pyrimidines derivatives, deoxythymidine, deoxycytosine or deoxyuridine. According to one embodiment of this aspect of the present invention, the repetitive core sequence is a mononucleotide sequence comprising deoxyguanine.

Initiator nucleic acid molecules of the present invention may be of any length provided, as mentioned that they are fully double stranded. Preferably, the minimum length of the initiator nucleic acid molecules is ten to twelve base pairs.

Synthesis of the initiator nucleic acid molecules may be affected by any method known in the art. The initiator nucleic acid molecules may be digested as double stranded molecules from natural (e.g., genomic or complementary DNA) or synthetic DNA (solid phase synthesized). Preferably a restriction enzyme that creates blunt ends is used for the digestion, so that the initiator nucleic acid molecules remain complementary along their entire length. If a restriction enzyme is used that creates 5′ or 3′ overhangs, a polymerase enzyme may be used to fill these in to create blunt ends. Specifically a polymerase with 5′ to 3′ polymerase activity can be used to fill in 5′ overhangs. In the case of 3′ overhangs, the 3′ to 5′ exonuclease activity present in some polymerases (especially T4 DNA polymerase) is required.

Alternatively, the initiator nucleic acid molecules may be the product of the de novo synthesis of the present invention.

Still alternatively, the initiator nucleic acid molecules may be the product of two hybridized single stranded DNA molecules. For example, the initiator nucleic acid molecules may be initially synthesized as single stranded oligonucleotide molecules utilizing solid phase chemistry, e.g. cyanoethyl phosphoramidite. Equipment and reagents for executing solid-phase synthesis are commercially available from, for example, Applied Biosystems.

Yet alternatively, the initiator nucleic acid molecules of this aspect of the present invention may be a combinatorial product of the above.

The single stranded oligonucleotides may be synthesized with modified bases and/or modifications at their 5′ end so long as the modifications do not affect the integrity of subsequent base pairing between the two complementary single stranded oligonucleotides.

Thus, as demonstrated in the Examples section below, the 5′ end of the single stranded oligonucleotides may be attached to a functional moiety which may be used for detection or anchoring to a solid support. Examples of such functional moieties include fluorescent molecules (such as tetramethylrhodamine (TAMRA) or Fluorescein (FLU)) or thiol groups without effecting synthesis of the homogeneous population of the nucleic acid molecules of the present invention. Other examples of functional moieties include, but are not limited to biotinylated molecules and disulfide molecules.

For example, the functional moiety may be integrated into a dUTP and subsequently incorporated on to the 5′ end of the initiator nucleic acid molecules using the enzyme terminal deoxynucleotidyl transferase (Life Technologies, Inc.). For example, fluorescent labeled dUTPs are commercially available such as fluorescein 12-dideoxyuridine-5′-triphosphate oligodeoxyribonucleotides—Boehringer Mannheim (Germany). Phosphorescent dUTPs may also be prepared [De Haas, R., 1999, Journal of Histochemistry and Cytochemistry, Vol. 47, 183-196] and used to produce phosphorescent labeled oligodeoxynucleotides.

Functional moieties may be covalently attached to the 5′ end of single stranded oligonucleotides via linker molecules. For example, Fidelity systems (Gaithersberg) provide custom-made oligonucleotides comprising a thiol functional group attached to its 5′-end by linker molecules of a variety of lengths. 5′-Thiol oligonucleotides are normally used in covalent attachment to a gold particle or film and in immobilization through p-maleimidophenyl isocyanate to a solid surface [Nicewarner Pena et al., JACS, 2002, 25, 7314; Peterson et al., Nucleic Acids Res., 2001, 24, 5163; Adessi et al., Nucleic Acids Res., 2000, 20, e87] and are therefore particularly useful for the field of nanoelectronics. Oligonucleotides linked at their 5′ end to TAMRA or Flu may be purchased, for example, from Alpha DNA (Montreal, Canada). The length of the linker molecule holding the functional groups can be varied to space the oligonucleotide away from the surface to overcome steric interference.

As mentioned above, the single stranded oligodeoxynucleotides may be synthesized using dNTPs with modified bases. It will be appreciated that the single stranded oligodeoxynucleotides may comprise bases that are not limited by the activity of a polymerase enzyme. Modified bases include but are not limited to synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No: 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. 5-methylcytosine substitutions have been shown to increase nucleic acid duplex stability by 0.6-1.2° C. [Sanghvi Y S et al. (1993) Antisense Research and Applications, CRC Press, Boca Raton 276-278].

Particularly useful, in the context of the present invention are those dNTPs that comprise metal chelating modified bases e.g. palladium chelating bases [M. Tasakam et al., Supramol. Chem. 13, 2001, 671] and copper chelating bases [H. Weizman, Y. Tor, J. Am. Chem. Soc. 123 (2001) 3375; E. Meggers et al., J. Am. Chem. Soc. 122 (2000) 10714] since this would enhance the natural conductivity of the product nucleic acid molecules.

Following synthesis and optional modification, the single stranded oligodeoxynucleotide molecules are purified, following which they are hybridized to produce the initiator nucleic acid molecules of the present invention. Exemplary conditions for hybridizing purified initiator nucleic acid molecules include incubation with complementary counterparts in 0.1 M KOH at a molar ratio of 1:1 for 15 minutes. According to the teachings of this aspect of the present invention there is a maximum length of single stranded oligodeoxynucleotide molecules that permits full hybridization. Thus, for the initiator nucleic acid molecule poly(dG)-poly(dC), the present inventors have found that single stranded oligodeoxynucleotides of thirty nucleotides hybridize to form 5′ overhangs and thus are responsible for the de novo synthesis of a non-homogenous population of nucleic acid molecules. Preferably the length of single stranded oligodeoxynucleotides which hybridized to form poly(dG)-poly(dC) is between ten and twenty nucleotides. The optimal length of complementary single stranded oligodeoxynucleotides for the production of initiator nucleic acid molecules of the present invention may be easily determined by one skilled in the art. Briefly, complementary single stranded oligodeoxynucleotides may be synthesized of various lengths and the homogeneous nature of the end-product nucleic acid molecules may be analyzed using various methods e.g. as described herein below.

Preferably, the alkali buffer is removed following hybridization of the single stranded oligodeoxynucleotide molecules e.g. by dialysis against 20 mM Tris-Acetate buffer, pH 7.0, for 4 hours.

The nucleic acid polymerase of the present invention is typically a DNA polymerase. Examples of DNA polymerases that can be used in accordance with this aspect of the present invention include, but are not limited to E. coli DNA polymerase I, the large proteolytic fragment of E. coli DNA polymerase I, commonly known as “Klenow” polymerase, “Taq” polymerase, T7 polymerase, Bst DNA polymerase, T4 polymerase, T5 polymerase and BCA polymerase. The DNA polymerase may lack 3′ to 5′exonuclease activity. According to the teachings of the present invention, the DNA polymerase is exonuclease free Klenow. DNA polymerases are available from a wide variety of manufacturers such as Fermentas (Lithuania), Promega, Sigma (Aldrich) and Biolabs (New England).

The concentration of initiator nucleic acid molecules is linearly correlated to the fully double stranded nucleic acid molecule product and its concentration is therefore selected according to the quantity required of fully double stranded nucleic acid molecule product. The concentration of nucleic acid polymerase and nucleotide triphosphates are selected according to both the quantity required of product fully double stranded nucleic acid molecule product and the length required of the fully double stranded nucleic acid molecule product. Thus, as demonstrated in the Examples section below, 0.2 μM of initiator may be added with 40 μg of Klenow polymerase and 1.5 mM nucleotides to synthesize a product of 10 kbase pairs.

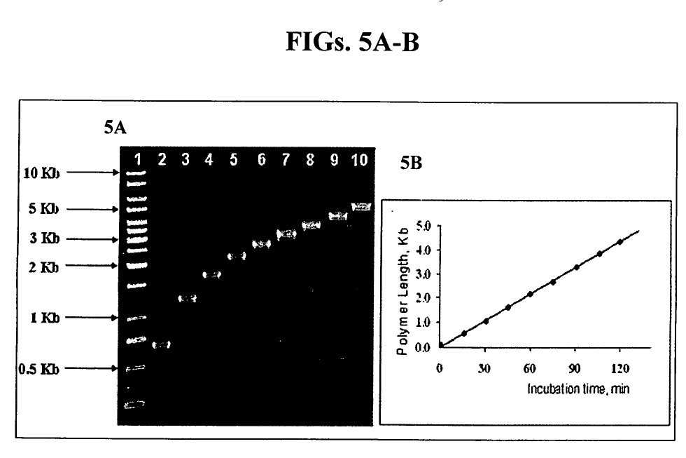

The initiator nucleic acid molecules, nucleotide triphosphates and nucleic acid polymerase are reacted for a time sufficient for polymerization (FIG. 5B). Typically, to synthesize a homogeneous population of nucleic acids 10 kbase pairs in length, a reaction time of several hours is required. The reaction is preferably effected at a non-denaturing temperature—e.g. between 25° C. and 37° C.

Other chemicals may also be added to the reaction to aid in the synthesis of the homogeneous population of nucleic acid molecules of the present invention. Exemplary chemicals include, but are not limited to potassium phosphate, magnesium chloride and DTT.

The reaction may optionally be terminated—e.g. by the addition of a chelator of divalent ions (e.g. EDTA) at any time.

Following their synthesis, the homogeneous population of nucleic acid molecules of the present invention may be isolated using such methods as electroelution, affinity chromatography, precipitation, column chromatography, and microfiltration. Alternatively, the product nucleic acid molecules may be isolated from a gel following electrophoresis using a commercially available kit such as those manufactured by Roche Applied Science, Indianapolis, and Promega Corporation, Madison.

In addition, following synthesis and optional isolation, the homogeneous nature and double-stranded structure of the population of nucleic acid molecules may be confirmed using methods described in Example 1 hereinbelow. For example, the population of nucleic acid molecules may be analyzed on an ethidium stained gel following electrophoresis. A homogeneous population of nucleic acid molecules should enter the gel and run as a single band (FIG. 1). The population of nucleic acid molecules may also be analyzed by size exclusion HPLC at high pH. At pH higher than 12.5, double stranded nucleic acid molecules separate into two single strands. The length of each strand may be analyzed by HPLC. As seen in FIG. 2A (solid line), poly(dG)-poly(dC) synthesized according to the teachings of the present invention was eluted as a single peak from the column at high pH, thus proving that G- and C-strands which compose the molecule were equal in size.

The above reagents of the present invention (e.g., initiator and nucleotides) may be packed in a kit for de novo synthesizing a homogeneous population of nucleic acid molecules. The kit of the present invention may, if desired, be presented in a pack which may contain one or more units of the kit of the present invention. The pack may be accompanied by instructions for using the kit. The pack may also be accommodated by a notice associated with the container in a form prescribed by a governmental agency regulating the manufacture, use or sale of laboratory supplements, which notice is reflective of approval by the agency of the form of the compositions.

According to one aspect, the kit comprises, preferably in a single container, deoxynucleotides, and the fully double stranded initiator nucleic acid molecules of the present invention.

Additionally, the kit may comprise polymerase preferably in a separate container. Other additional agents may be comprised in the kit such as magnesium chloride, potassium phosphate, EDTA and DTT.

An example of a homogeneous population of fully double stranded nucleic acid molecules generated according to the above teachings are preferably each at least 100 base pairs long and have the same repetitive core sequence selected from the group consisting of mono-, di-, or tri-nucleotide, with the proviso that the repetitive core sequence is not A mononucleotide.

A further example of a homogeneous population of fully double stranded nucleic acid molecules generated according to the above teachings are preferably each at least 1000 base pairs long and have the same repetitive core sequence of an A mononucleotide (i.e. poly(dA)-poly(dT).

Typically, the synthesized molecules are no longer than 10 kbase pairs. According to a preferred embodiment of the present invention, the homogeneous population of fully double stranded nucleic acid molecules comprises poly(dG)-poly(dC).

According to a particular embodiment of the present invention, the homogenous population of fully double stranded nucleic acid molecules may be immobilized onto a solid support.

Solid supports may be comprised of any material including but not limited to conducting materials, semiconducting materials, thermoelectric materials, magnetic materials, light-emitting materials, biominerals and polymers.

According to this aspect of the present invention, the conducting material may be a metal, such as a transition metal. Examples of transition metals include, but are not limited to silver, gold, copper, platinum, nickel and palladium.

Examples of semiconducting materials that may be used as solid supports include, but are not limited to a group IV semiconducting material, a group II-VI semiconducting material and a group III-V semiconducting material. As used herein, the term “Group” is given its usual definition as understood by one of ordinary skill in the art. For instance, Group II elements include Zn, Cd and Hg; Group III elements include B, Al, Ga, In and Tl; Group IV elements include C, Si, Ge, Sn and Pb; Group V elements include N, P, As, Sb and Bi; and Group VI elements include O, S, Se, Te and Po.

The magnetic material may be any magnetic material such as a paramagnetic material or a ferromagnetic material. Examples of paramagnetic materials that can be used according to this aspect of the present invention include, but are not limited to aluminum, copper, and platinum. Examples of ferromagnetic materials that can be used according to this aspect of the present invention include, but are not limited to magnetite, cobalt, nickel and iron.

Examples of light-emitting materials that may be used according to this aspect of the present invention include, but are not limited to dysprosium, europium, terbium, ruthenium, thulium, neodymium, erbium, ytterbium and any organic complex thereof.

An example of a biomineral that may be used according to this aspect of the present invention is calcium carbonate.

Examples of polymers that may be used according to this aspect of the present invention include, but are not limited to polyethylene, polystyrene and polyvinyl chloride.

Examples of thermoelectric materials that may be used according to this aspect of the present invention include, but are not limited to bismuth telluride, bismuth selenide, bismuth antimony telluride and bismuth selenium telluride.

Immobilization of the homogenous population of nucleic acids of the present invention on to a solid support may be effected directly (e.g. by irradiation) or via a functional moiety attached to the 5′ end of the nucleic acid molecules of the present invention as described hereinabove. The solid support may also comprise a carrier which binds to the nucleic acid molecules of the present invention. The carrier may be coated onto the solid surface as a film using known methods such as spraying, dipping, brushing, stamping, vapor deposition, and coating using a film coater. The carrier may be physically adsorbed to a solid surface, or chemically carried through a covalent bond or the like. The carrier may be carried on the whole surface of the substrate, or may be carried on a part of the surface, as required.

For example, the method of DNA immobilization and patterning by electrostatic interactions with a cationic bilayer adsorbed to a self assembled monolayer (SAM) can be applied. As a result, the cationic lipids readily form layers on self assembled alkyl thiols possessing terminal carboxylic groups. DNA then can be electrostatically connected to the cationic layer [Schouten, S., Stroeve, P. and Longo, L. M. (1999) Langmuir, 15, 8133-8139].

Another method of immobilizing DNA uses the combinatorial photolitographic approach developed originally by Affimetrix. Briefly, the method involves illumination through a micro structured photo-mask of a chip modified with photolabile protection groups that creates selected areas to which phosphoramidate building blocks can be attached for the sake of further nucleotide attachment. [Niemeyer, C. M. and Blohm D (1999). Angew. Chem., 38 No.19, 2865-2869].

Further methods involve utilizing glass surfaces coated with the carrier 3-mercaptosylane for the attachment of 5′-disulfide modified DNA molecules via disulfide bonds (Rogers, Y. H., Baucom, P. J., Huang, Z. J., Bogdanov, V., Anderson, S. and Boyce, M. T. (1999), Analytical Biochemistry 259, 31-41). Another method involves immobilizing activated DNA on aldehyde containing polyacrylamide gels for preparation of MAGIChips, which are microarrays of gel immobilized compounds on a chip [Proudnikov, D., Timofeev, E. and Mirzabekov, A. (1998) Analytical Biochemistry 259, 34-41].

By way of another example, it has been shown that semi conducting materials coated with an amine carrier can immobilize nucleic acid molecules comprising a 5′ linked thiol moiety [Strother et al., Nucleic Acids Research, 2000, Vol. 28, No. 18 3535-3541].

Another exemplary system is the use of avidin as a carrier on a solid surface to immobilize nucleic acid molecules comprising a 5′ linked biotin moiety on a particle surface coated with avidin [Alivisatos, K. P. et al., Nature 382, 609, 1996].

Yet another exemplary system is the use of short single stranded DNA molecules as a carrier on a solid surface to immobilize the nucleic acid molecules of the present invention comprising a 5′ linked complementary single stranded moiety.

The homogeneous population of fully double stranded nucleic acid molecules may also be metallized. Metal ions may be comprised within the nucleic acid structure itself i.e. between the bases or attached to the DNA backbone (see e.g. Richter J., Physica E 16 (2003) 157-173) so as to enhance its native conducting properties.

For example, metal may be grown on the homogenous population of nucleic acid molecules of the present invention by a process known as seeding. This process initially involves the binding and subsequent activating of DNA by metal complexes. This activation process is crucial in establishing as many metal seeds as possible onto the molecule. Several seed metals may be used including various platinum and palladium complexes, and metal ions such as silver and cadmium. In general, platinum and palladium complexes bind to the bases whereas the metal ions bind to the backbone via electrostatic forces. Next, the bound seeds are typically treated with a reducing agent. This procedure is especially successful for seed ions but is also helpful in the case of platinum complexes. Examples of reducing agents which may be used according to this aspect of the present invention include, but are not limited to dimethylaminoborane, hydroquinone and sodium borohydride. The agent may be buffered to slow down the reaction and to prevent unwanted cluster growth in solution. Finally, in the third step, metal solutions and reducing agents are introduced to the chemically activated nucleic acid molecules of the present invention. The metal solutions may comprise different or identical metals to those originally used for seeding. This promotes immediate cluster growth, as the metal seeds on the template serve as catalysts for further reduction of metal. In this autocatalytic process metal complexes or ions from solution are preferably reduced on already reduced metal. Depending on the time allowed for reaction and the concentrations of the metal solution and reducing agent, different stages of metallization can be achieved.

Other methods known in the art may also be used to metallize the homogeneous fully double-stranded nucleic acid population of the present invention such as those described by Aich, P. et al., J. Mol. Biol. 294 (1999),477 and Patolsky F. et al., Chem. Int. Ed. Engl. 41 (2002) 2323.

Semiconducting ions may also be attached to the homogenous population of fully double-stranded nucleic acid molecules of the present invention using any method such as those described in the literature [T. Torimoto, et al., J. Phys. Chem. B 103 (1999) 8799; X. D. Zhang, et al., Acta Chim. Sin. 60 (2002) 532.; S. R. Bigham, J. L. CoJer, Colloid Surf. A 95 (1995) 211; J. L. CoJer, et al., Appl. Phys. Lett. 69 (1996) 3851].

Sequence specific metallization, is also envisaged in the scope of the present invention. As mentioned hereinabove, the homogenous population of fully double-stranded nucleic acid molecules of the present invention may comprise a functional moiety at their 5′ end. The functional moiety may be a particular sequence of single stranded DNA which is a target for sequence specific metallization. An exemplary method of sequence specific metallization is that described by Keren et al., [Science 297 (2002) 72].

Alternatively, or additionally the homogenous population of fully double-stranded nucleic acid molecules of the present invention may be doped with oxygen so as to enhance its conducting properties. For example, oxygen doping of poly(dG)-poly(dC) was shown to increase its conductance by several orders of magnitude [H Y Lee et al., Applied Physics Letts, 80, 9, 2002]. The homogenous population of nucleic acid molecules of the present invention can be doped, for example, differentially along their length, or radially, and either in terms of identity of dopant, concentration of dopant, or both. This may be used to provide both n-type and p-type conductivity in a single item.

The homogeneous population of fully double-stranded nucleic acid molecules of the present invention complexed with photo-sensitive dyes may be used in the field of micro- and sub-microelectronic circuitry and devices. Thus for example, the homogeneous population of nucleic acid molecules may be used as wires, either as single molecules or as bundles of molecules, also referred to herein as fibers. The wires of the present invention may additionally comprise insulating materials (e.g. polyvinylchloride, PVC) surrounding the wires.

The current-voltage characteristics of the wires of the present invention may be determined by attaching to planar electrodes that are fabricated on an insulating surface (SiO₂ or SiN₄). These measurements are performed at temperatures ranging from room temperature down to cryogenic temperatures and at various environmental conditions (air, solvent and vacuum).

The wires of the present invention may be connected to other molecular wires such that the interconnected wires may conduct electricity through them. For example, linking can be provided through oxidized thiol groups. Alternatively, amino modified nucleotides can be attached to the 5′ phosphates using standard phosphoramidate chemistry. Alternatively, the homogeneous population of nucleic acid molecules of the present invention can be reacted with carbonyldiimidazole and a diamine to yield a phosphoramidate that has a free primary amine. This amine can then be reacted with nucleotides modified with amino reactive groups.

It will be appreciated, though, that the interconnected wires of the present invention can be of infinite length (i.e., macroscopic fibrous structures) and as such can be used in the fabrication of hyper-strong materials.

In addition the wires of the present invention may be connected to a solid support as described hereinabove to produce an electronic circuit.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Synthesis and Characterization of poly(dG)-poly(dC)

Materials and Methods

Materials: Unless otherwise stated, reagents were obtained from Sigma-Aldrich (USA) and were used without further purification. 2′-Deoxyribonucleoside 5′-triphosphates (dGTP and dCTP) were purchased from Sigma-Aldrich (USA). Esherichia coli Kienow fragment exonuclease minus (i.e. lacking 3′ to 5′ exonuclease activity was purchased from Fermentas (Lithuania).

DNA samples: Oligonucleotides were purchased from Alpha DNA (Montreal, Canada). Fluorescein-(Flu) and tetramethylrhodamine-(TAMRA) labeled oligonucleotides were also purchased from Alpha DNA (Canada). The Flu and TAMRA labels were linked to the terminal base at the 5′-end of G-12-mer (dG)₁₂ and C-12-mer (dC)₁₂ oligonucleotides, correspondingly, via a six-carbon linker. The nomenclature used for the above oligonucleotides is as follows: Flu-(dG)₁₂, TAMRA-(dC)₁₂. Poly(dC)-oligonucleotides were purified using an ion-exchange Western Analytical Products (USA) PolyWax LP column (4.6×200 mm, 5 μm, 300 Å) at pH 7.5. Poly(dG)-oligonucleotides were purified using a ion-exchange HiTrap QHP column (1×5 ml) from Amersham-Biosciences (Sweden) in 0.1M KOH. The dye-labeled probes were purified by HPLC using a Vydac (USA) reverse-phase C₁₈ column (4.6×250 mm). HPLC purified oligonucleotides were desalted using pre-packed Sephadex G-25 DNA-Grade columns (Amersham-Biosciences, Sweden). Purified oligomers were incubated with their complementary counterparts in 0.1 M KOH at a molar ratio of 1:1 for 15 minutes and dialyzed against 20 mM Tris-Acetate buffer, pH 7.0, for 4 hours. All oligonucleotides were quantified spectrophotometrically using their respective extinction coefficients. Concentrations of G- and C-homopolymers were calculated using extinction coefficients at 260 nm of 11.7 and 7.5 mM⁻¹ cm⁻¹ for G and C bases. CD spectra of poly(dG)-poly(dC) were measured on Aviv Model 202 series (Aviv Instrument Inc., USA) Circular Dichroism Spectrometer. Each spectrum was recorded from 220 to 340 nm and was an average of 5 measurements.

DNA Polymerase assay: A standard reaction contained 60 mM KPi, pH 7.4, 5 mM MgCl₂, 5 mM dithiothreitol (DTT), and 1.5 mM each of dCTP and dGTP, the Kienow exo⁻ and template-primer. The concentration and nature of template-primer and concentration of Klenow exo⁻ were as described in the Figure legends. The reaction was started by the addition of the enzyme. The incubation was at room temperature (25° C.) or at 37° C. for 2-4 hours. The reaction was terminated by the addition of EDTA to a final concentration of 10 mM. Reaction products were analyzed by size exclusion and ion-exchange HPLC, as well as by electrophoresis on an agarose gel.

Gel Electrophoresis: The products of polymerase synthesis and commercial preparations of poly(dG)-poly(dC) (Sigma, lot 103K10561) were loaded onto a 1% agarose gel and then electrophoresed at room temperature at 130 volts for 1 hour. TAE buffer, in addition to being used to prepare the agarose, also served as the running buffer. The dimensions of the agarose gel were 10×10 cm with 2×4 mm 14-wells. The gel was stained with ethidium bromide (5 μg/ml) and visualized with a Bio Imaging System 202D (302 nm).

HPLC separation of the polymerase products: Poly(dG)-poly(dC) was separated from nucleotides, enzyme and other reaction components of the synthesis using size-exclusion HPLC. The separation was achieved with a TSK-gel G-DNA-PW HPLC column (7.8×300 mm) from TosoHaas (Japan) by isocratic elution with 20 mM Tris-Acetate, pH 7.0, for 30 minutes at a flow rate of 0.5 ml/min. Size-dependent separation of the strands composing poly(dG)-poly(dC) was performed using the same column by isocratic elution with 0.1 M KOH at a flow rate of 0.5 ml/min. The injection volumes were 50-200 μl. All experiments were conducted on an Agilent 1100 HPLC system with a photodiode array detector. Peaks were identified from their retention times obtained from the absorbance at 260 nm. Data were collected from PDA and analyzed by Microsoft Excel.

Separation of 10-18 base pair long G- and C-homopolymers originating from the early polymerase synthesis was performed using ion-exchange TSKgel DNA-NPR column (4.6×75 mm) from Tosoh Biosciences (Japan) at high pH. The oligonucleotides were eluted with a linear gradient of KCl from 0 to 1 M in 0.1 M KOH at a flow rate of 0.6 ml/min. Ion-exchange HPLC was also used as a method to determine the concentrations of dGTP and dCTP nucleotides in the assay. HPLC separation of the dNTPs was performed with an ion-exchange PolyWax LP HPLC column (4.6×200 mm, 5 μm, 300 Å) from Western Analytical (USA), using a linear gradient of 20 to 500 mM potassium phosphate buffer, pH 7.4.

Results

Electrophoresis analysis of poly(dG)-poly(dC) preparations both purchased from Sigma and synthesized by Kienow exo⁻ by the present inventor, in a non-denaturing agarose gel, is shown in FIG. 1. As seen in this figure the commercial preparation does not enter the gel (lane 2). This might be due to aggregation of the DNA molecules. Heating the preparation for 30 minutes at 70° C. did not result in dissociation of the aggregates. A small fraction of the molecules which entered the gel following heat treatment may be characterized by broad distribution of molecular sizes and shows a smeared band pattern (FIG. 1, lane 3). In contrast to the commercial polymer, poly(dG)-poly(dC) synthesized by the present inventors entered the gel and was characterized by a narrow distribution of molecular sizes (FIG. 1 lane 4).

Analysis of strands which compose the commercial and synthesized polymers was performed by size exclusion HPLC at high pH. At pH higher than 12.5, the poly(dG)- and the poly(dC) strands are separated. As seen in FIG. 2A (solid line), poly(dG)-poly(dC) synthesized by the present inventors is eluted as a single peak from the column at high pH, thus proving that G- and C-strands which compose the polymer are equal in size. Elution profile of the commercial polymer is different from the synthesized one and is presented by two overlapped peaks (FIG. 2A, dashed line). Absorption spectroscopy analysis of the eluted fraction showed that the earlier peak eluted between 13 and 16.5 minutes is characterized by spectrum of C-homopolymer, while the peak eluted between 17 and 22 minutes has a spectrum of G-homopolymer (FIGS. 2B-C). Different retention times of C- and G-strands are indicative of different lengths of the strands composing the commercial polymer. C-strand of the commercial polymer is eluted from the column in a volume similar to that of the 7 Kbase pairs poly(dG)-poly(dC) (FIG. 2A). The G-strand is eluted in a volume corresponding to that of 1.5 Kbase pairs DNA (data not shown). The above analysis clearly shows that the G-strand composing poly(dG)-poly(dC) obtained from Sigma is about five times shorter than the corresponding C-strand.

Poly(dG)-poly(dC) was synthesized by the present inventors using the Klenow exo⁻ fragment of DNA polymerase I in the presence of dGTP, dCTP and (dG)₁₀-(dC)₁₀ oligonucleotides. If primed by non-purified (dG)₁₀-(dC)₁₀, the synthesis yielded polymer molecules with large length variability (lane 5, FIG. 1). (dG)₁₀-(dC)₁₀ prepared from HPLC purified (dG)₁₀ and (dC)₁₀, as described above, primed synthesis of uniform poly(dG)-poly(dC) (FIG. 1, lane 4).

It has been shown that (dG)₃₀-(dC)₃₀ can also efficiently prime synthesis of long poly(dG)-poly(dC). However, this synthesis does not yield uniform poly(dG)-poly(dC), regardless of whether the oligonucleotides composing the template-primer are purified by HPLC or not. This might be due to formation of kinetically stable structures with overhangs when 30 base (or longer) poly(dG) and poly(dC) oligonucleotides are used to form a double stranded template-primer. When primed by overhangs containing, (dG)₁₂-(dC)₁₅ and (dG)₁₅-(dC)₁₂ duplexes, the synthesis yielded various lengths of poly(dG)-poly(dC) (data not shown), thus supporting the above suggestion. Overhangs containing temporary structures, even if formed while annealing of (dG)₁₀ and (dC)₁₀, are spontaneously and rapidly rearranged (at 37° C.) into more stable, completely annealed (dG)₁₀-(dC)₁₀ duplexes that prime synthesis of uniform poly(dG)-poly(dC).

To estimate the content of dG and dC bases in poly(dG)-poly(dC), the amount of nucleotides consumed during the synthesis of the polymers was calculated. The reaction was conducted as described above and was arrested by addition of EDTA to the assay mixture. The products of the synthesis were separated from dGTP and dCTP by size-exclusion HPLC. As seen in FIG. 3A, incubation of Klenow exo⁻ with dGTP, dCTP and (dG)₁₀-(dC)₁₀ resulted in the appearance of a peak which eluted prior to total column volume. This peak corresponds with a high molecular weight poly(dG)-poly(dC) product of the synthesis. Its position shifted left and its height grew (FIG. 3A) as the synthesis progressed. The peak eluted from the column in total volume comprised a mixture of dGTP and dCTP. The height of the latter peak decreased together with the increase of the poly(dG)-poly(dC) peak (FIG. 3A), which corresponded with incorporation of the nucleotides into the polymer. The peak eluted with total volume was collected and amounts of dGTP and dCTP in the peak were estimated. dGTP and dCTP were separated one from another by ion-exchange RPLC as shown in FIG. 3B. The first peak eluted from the ion-exchange column corresponded to dCTP and the second one to dGTP. Both peaks were collected separately and quantities of the nucleotides were estimated by spectrophotometer; 7.5×10³ and 11.7×10³ M⁻¹ cm⁻¹ extinctions coefficient at 260 nm were used for dCTP and dGTP correspondingly. The results of this analysis are summarized in Table I below, all values being an average of 5 measurements.

TABLE 1

| Time of synthesis, min | dGTP, nmoles | dCTP, nmoles |
|---|---|---|
| 0 | 18.3 | 18.4 |
| 30 | 15.8 | 16.1 |
| 60 | 10.5 | 11.0 |
| 120 | 8.6 | 8.5 |
| 150 | 6.3 | 6.4 |

As evident from the table, equal amounts of dCTP and dGTP were consumed from the assay during synthesis of the polymer. Thus, the data presented in FIGS. 2 and 3 suggest that the procedure described herein results in the formation of a one-to-one double helical complex of polydeoxyguanylate and polydeoxycytidylate.

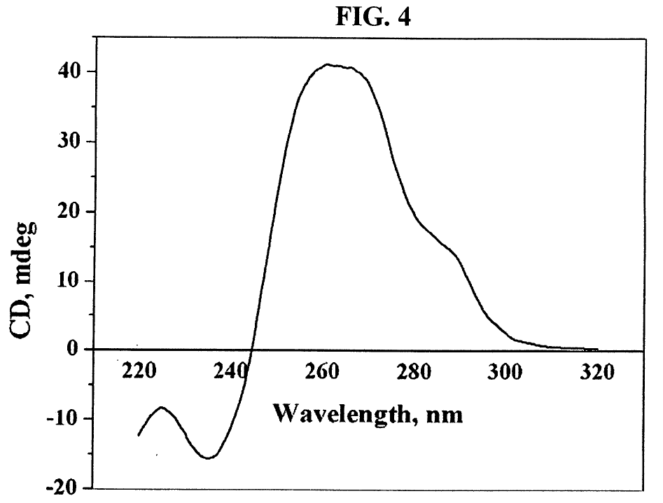

Additional evidences for the double stranded nature of synthesized poly(dG)-poly(dC) come from digestion experiments of the polymer with Deoxyribonuclease I (DNase) and from the CD spectroscopy. DNase efficiently digests poly(dG)-poly(dC) to short oligonucleotides. The enzyme is specific with respect to double-stranded DNA; thus digestion by the enzyme is reflective of the double stranded nature of the polymer. The major characteristics of CD spectrum of poly(dG)-poly(dC), namely a positive bend at 235 nm, a crossover at 244, and negative band at 235 nm, are similar to those reported for double stranded poly(dG)-poly(dC) by Grey D. M [Biopolymers, 13, 1974, 2087-2102]. A typical spectrum for a 3 kbp poly(dG)-poly(dC) is shown in FIG. 4.

### Example 2

### Determination of the Mechanism of Synthesis of poly(dG)-poly(dC) by Klenow⁻ exo Fragment

Materials and Methods

Mass spectrometer conditions: Mass spectrometric measurements of oligonucleotides were carried out on a Finnigan LCQ Classic ion trap instrument (ThermoFinnigan, San Jose, Calif.) equipped with its standard heated capillary electrospray source. The source was operated in the negative ion mode, with a heated capillary temperature normally set at 150° C. and needle voltage at −3 kV [Huber et al., 2000, J Mass Spectrom., 35, 870-877]. Mass spectra were recorded in a row scan mode in the mass range from m/z of 500 to 2000. All mass spectra were obtained by signal averaging for 1 minute at a scan rate of 3 microscans/scan. Solutions of oligonucleotides were admitted by direct infusion with a 100 μl Hamilton gas-tight syringe (Holliston, Mass.) at a flow rate of 3 μg/min. Typically, 1 μM solution of oligonucleotide was injected into 25 mM triethylamine (TEA), 25 mM hexafluoroisopropanol (HFIP) and 50% acetonitrile.

FRET measurements: Extension of fluorescently labeled oligonucleotides was performed in 100 mM Tris-Acetate, pH 8.0, 3 mM MgCl₂, 5 mM DTT, and 1 mM each of dCTP and dGTP, 0.8 μg/ml Klenow exo⁻ and 5 μM Flu-(dG)₁₂-TAMRA-(dC)₁₂ duplex. The steady-state fluorescence measurements were performed with Model LS50B Perkin-Elmer (England) Luminescence Spectrometer. Excitation was at 490 nm with emission at 520 nm. The slits for excitation and emission monochromators were both set at 2.5 and 2.5 mm.

Absorption spectra of the synthesized products were recorded with U2000 Hitachi (Japan) spectrophotometer. The contents of Flu, TAMRA and G-C base pairs were estimated using the following extinction coefficients: ε^{Flu} (494 nm)=77,000 M⁻¹ cm⁻¹, ε^{TAMRA} (558 nm)=90,000 M⁻¹ cm⁻¹ (16), ε^{GC} (260 nm)=14,800 M⁻¹ cm⁻¹ (4). The contributions of the dyes to absorption at 260 nm were calculated based on their concentrations and their extinction coefficients, ε^{Flu}=20,900 M⁻¹ cm⁻¹; ε^{TAMRA}=31,900 M⁻¹ cm⁻¹ at 260 nm. The contribution of the dye at 260 nm was subtracted and the concentration of G-C pairs in each sample of synthesized poly(dG)-poly(dC) was then determined.

Results

The kinetics of poly(dG)-poly(dC) synthesis primed by HPLC purified (dG)₁₀-(dC)₁₀ is depicted in FIGS. 5A-B. The molecules grew continuously until the dGTP and dCTP were exhausted. Analysis of the data reveals linear dependence of the polymer length on time of synthesis (FIG. 5B). Thus, the rate of polymer growth is independent of the length of the fragments being synthesized. The reaction product could be purified and used as a template-primer for a further synthesis ultimately leading to the production of thousand base pair long uniform molecules (data not shown).

The effect of modifications on the ability of (dG)₁₂-(dC)₁₂ to prime synthesis of poly(dG)-poly(dC) is summarized in Table 2 below.

TABLE 2

| | | |
|---|---|---|
| | SEQ | Priming of |
| | ID | poly(dG)-poly(dC) |
| Oligonucleotide | NO: | synthesis |

| | | |
|---|---|---|
| 5′-GGGGGGGGGGGGA-3′ | 1 | No |
| −5′-CCCCCCCCCCCCA-3′ | 2 | |
| 5′-AGGGGGGGGGGGG-3′ | 3 | Yes |
| −5′ACCCCCCCCCCCC-3′ | 4 | |
| 5′-GGGGGGGGGGGGA-3′ | 1 | No |
| −5′-CCCCCCCCCCCCC-3′ | 5 | |
| 5′-GGGGGGGGGGGGG-3′ | 6 | Yes |
| −5′-ACCCCCCCCCCCC-3′ | 4 | |
| 5′-GGGGGGGGGGGGG-3′ | 6 | No |
| −5′-CCCCCCCCCCCCA-3′ | 2 | |
| 5′-AGGGGGGGGGGGG-3′ | 3 | Yes |
| −5′-CCCCCCCCCCCCC-3′ | 5 | |
| 5′-Flu-GGGGGGGGGGGG-3′ | 7 | Yes |
| −5′-TAMRACCCCCCCCCCCCC-3′ | 8 | |
| 5′-NH2-GGGGGGGGGGGG-3′ | 9 | Yes |
| −5′-NH2-CCCCCCCCCCCCC-3′ | 10 | |
| 5′-GGGGGGGGGGGG-NH2-3′ | 11 | No |
| −5′-CCCCCCCCCCCCC-NH2-3′ | 12 | |
| 5′-SH-GGGGGGGGGGGG-3′ | 13 | Yes |
| −5′-SH-CCCCCCCCCCCCC-3′ | 14 | |

Table 2 continued

The data presented in Table 2 shows that either covalent modification of one of the two 3′-ends of (dG)₁₂-(dC)₁₂ or substitution of C or (and) G at the 3′-end(s) of the oligonucleotide with A-nucleotide resulted in a complete loss of the oligonucleotide ability to prime the synthesis. In contrast, covalent modification of the 5′-ends or replacement of either C or G bases at the 5′-ends with A-nucleotide had no effect on the capacity of the oligonucleotide to prime the synthesis. This fact allowed the dynamics of the polymerase synthesis by fluorescence resonance energy transfer (FRET) using (dG)₁₂-(dC)₁₂ labeled at the 5′-ends with different fluorescent dyes to be studied.

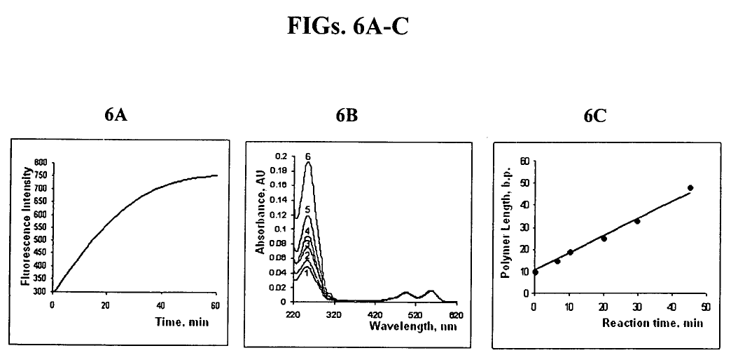

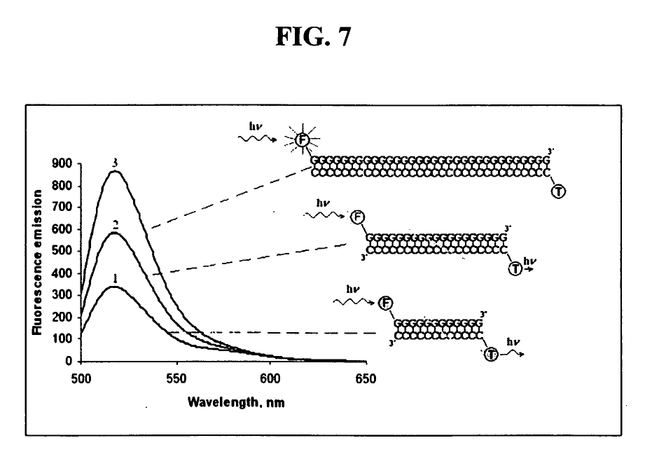

In FRET, a donor fluorophore is excited by incident light, and if an acceptor is in close proximity, the excited state energy from the donor is transferred by means of intermolecular long-range dipole-dipole coupling. The efficiency of FRET is dependent on the inverse sixth power of the intermolecular separation the donor. Thus, FRET provides a very sensitive measure of small changes in intermolecular distances. Flu energy donor and TAMRA energy acceptor moieties meet spectroscopic criteria important in a study of energy transfer [Wu P, 1994, Anal. Biochem., 218, 1-13]. The above dyes were employed in this work to monitor dynamics of the primer-template extension by Polymerase. (dC)₁₂ and (dG)₁₂ oligonucleotides labeled at the 5′-ends with TAMRA and Flu correspondently were used in FRET experiments. The oligonucleotides were purified by HPLC and annealed as described above. Emission of Flu in the (dG)₁₂-(dC)₁₂ oligonucleotide labeled at the opposite 5′-ends with Flu and TAMRA was strongly quenched compared to that of the Flu-(dG)₁₂. Quenching was independent of concentration of the (dG)₁₂-(dC)₁₂ oligonucleotide thus supporting the intramolecular mechanism of excitation energy transfer from Flu to TAMRA. Addition of Klenow exo⁻ to the assay mixture containing Flu-(dG)₁₂-(dC)₁₂-TAMRA, dGTP and dCTP caused the increase of Flu emission in time (FIG. 6A). Aliquots were withdrawn from the assay at different times and the products of the synthesis were analyzed by absorption spectroscopy. Spectra of the oligonucleotide and products of 5, 10, 20, 30, and 40 minute syntheses are shown in FIG. 6B. Peaks at 565, 496, and 260 nm are attributed to TAMRA, Flu and DNA respectively. The peak at 260 nm is mainly due to absorption of the oligonucleotide, whereas contribution of both the dyes to the absorption at 260 nm is minor. Using extinction coefficient of 14.8×10³ M⁻¹cm⁻¹ for a G-C base pair at 260 nm, the average number of base pairs in the products of the synthesis was calculated. The dependence of the calculated length of poly(dG)-poly(dC) on the time of synthesis is shown in FIG. 6C. Fluorescence emission spectra of the products of Flu-(dG)₁₂-(dC)₁₂-TAMRA extension are presented in FIG. 7, together with schematic presentation of structures of the double-labeled products of the extension. Energy transfer between the dyes attached at both sides of the template-primer was apparent as a decrease in the contribution of the Flu donor and an increase in the relative contribution of the TAMRA acceptor. The extension resulted in an increase of the separation distance between the 5′-ends of the strands and in a loss of the ability of Flu and TAMRA to communicate via FRET. When the length of extended polymer reached approximately 30 base pairs (FIGS. 6 and 7), no communication of the dyes was seen and Flu emission reached maximum levels. The latter is in good agreement with the FRET theory, saying that no energy transfer can be observed at distances greater than 100 Å [Stryer L., 1978, Annu. Rev. Biochem., 47, 819-846]. The data of FRET analysis presented in FIGS. 6 and 7 clearly show that the 5′-ends are moving in opposite directions during extension of the template-primer by Klenow exo⁻.

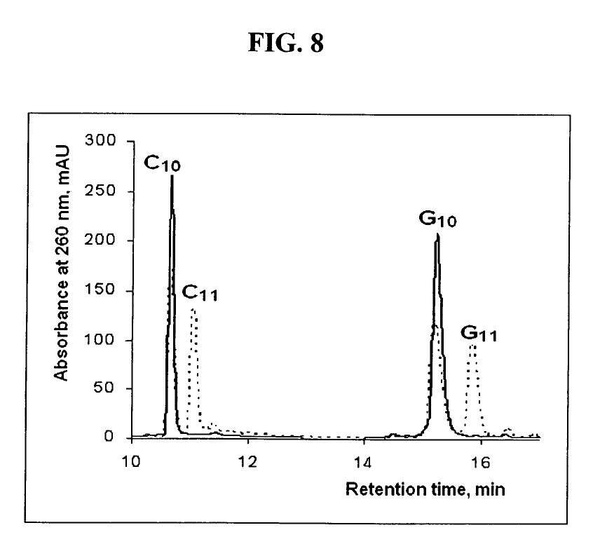

To investigate the mechanism of synthesis in more detail, early synthesis products were analyzed by a combination of HPLC and Mass spectroscopy. The synthesis was conducted for 5 minutes at 37° C. in the presence of small amounts of Klenow exo⁻, dGTP, dCTP, and (dG)₁₀-(dC)₁₀. Products of the synthesis were separated from the nucleotides and passed through the ion-exchange HPLC column at high pH. Anion-exchange HPLC at high pH enables separation of G- and C-strands composing the template-primer. G-bases undergo complete deprotonation at pH higher than 12 and an additional negative charge is introduced to each base of G-strand. Higher negative charge of G-strand compared to corresponding C-strand results in tighter binding of the G-strand to a positively charged matrix of the column, and as a result in its elution from the column at higher salt concentrations. FIG. 8 presents data of ion exchange HPLC of (dG)₁₀-(dC)₁₀ (continuous curve) and products of 5 minute syntheses (dashed curve). Molecular masses of oligonucleotides composing the template-primer and eluted in the first and second peaks (solid curve) have been estimated by Mass spectroscopy to be equal to 2868 and 3228D. The estimated masses correspond well with (dC)₁₀and (dG)₁₀. Incubation of (dG)₁₀-(dC)₁₀ with Klenow ex⁻, dGTP and dCTP for 5 minutes results in appearance of two new peaks on the chromatogram to the right of (dC)₁₀ and (dG)₁₀ ones (FIG. 8, dashed curve). Molecular masses of oligonucleotides eluted in the new peaks are equal to 3117 and 3558, which correspond with masses of (dC)₁₀ and (dG)₁₀. These data demonstrate that an elementary step of (dG)₁₀-(dC)₁₀ extension includes addition of one base to each of the strands composing the oligonucleotide. As seen in FIG. 8 (dashed curve), incubation for 5 minutes of 15 μM (dG)₁₀-(dC)₁₀ with 2 μM Klenow ex⁻ at 37° C. results in conversion of approximately 45% of the oligonucleotide to product. Based on these data, a turnover number of 60 min-1 for Klenow ex⁻ in the reaction of (dG)₁₀-(dC)₁₀ was calculated as summarized in Table 3 below.

TABLE 3

| | | | |
|---|---|---|---|
| | SEQ | | |
| | ID | | *TN |
| Template - primer | NO: | Product of extension | min⁻¹ |

| | | | |
|---|---|---|---|
| 5′GGGGGGGGGG3′- | 15 | 5′GGGGGGGGGGG3′- | 60 |
| 5′CCCCGCCCCC3′ | 16 | 5′CCCCCCCCCCC3′ | |
| 5′GGTGGGGGGGGGA3′- | 17 | 5′GGTGGGGGGGGGA3′- | 50 |
| 5′GCCCCCCCCCCCA3′ | 2 | 5′GCCCCCCCCCCCACC3′ | |
| 5′GGGTGGGGGGGGA3′- | 18 | 5′GGGTGGGGGGGGA3′- | 20 |
| 5′CCCGCCCCCCCCA3′ | 2 | 5′CGCCCCCCGCGCACCC3′ | |
| 5′GGGGTGGGGGGGA3′- | 19 | 5′GGGGTGGGGGGGA3′- | 5.6 |
| 5′CCCCCCCCCCCCA3′ | 2 | 5′CCCCCCCCCCCGACCCC3′ | |
| 5′GGGGGTGGGGGGA3′- | 20 | 5′GGGGGTGGGGGGA3′- | 0.3 |
| 5′CCCCCCCCCCCCA3′ | 2 | 5′CCCCCCCCCCCCACCCCC3′ | |

*Rate is expressed as number of the enzyme's turnovers per minute.

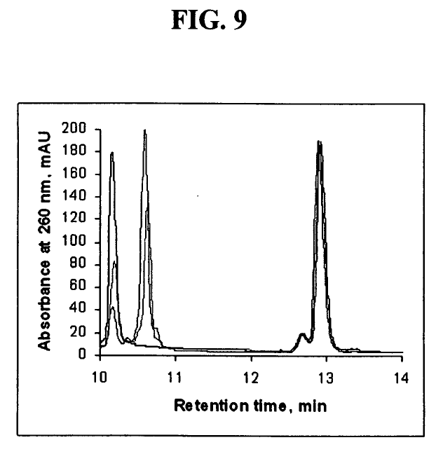

Replacement of C or G, even at one of the 3′-ends of (dG)₁₂-(dC)₁₂ with A-nucleotide, resulted in complete loss of the oligonucleotide ability to prime the synthesis (Table 2). A reason for that might be the inability of the enzyme to properly pair the A-nucleotide at the 3′-end if no T-nucleotides are present in the sequence of the complimentary strand. Indeed, when T-nucleotide was introduced into a sequence of the strand, replication was restored. Expansion of the double stranded 5′CCCCCCCCCCCCA3′-5′GGGGTGGGGGGGA3′ by Klenow exo⁻ is demonstrated in FIG. 9. Strands composing the oligonucleotide were purified to homogeneity by HPLC, and annealed. As seen in FIG. 9, incubation of 5′CCCCCCCCCCCCA3′ (SEQ ID NO: 2) and 5′GGGGTGGGGGGGA3′ (SEQ ID NO: 20) with Klenow exo⁻, dGTP and dCTP results in extension of 5′CCCCCCCCCCCCA3′-strand. This is seen as a shift of the peak corresponding to 5′CCCCCCCCCCCCA3′ on the chromatogram (see FIG. 8, red and blue curves). The position of the second peak, corresponding to 5′GGGGTGGGGGGGA3′, remains unchanged. Molecular mass of the oligonucleotide eluted in the shifted peak has been estimated by Mass spectroscopy (see Materials and Methods) to be equal to 4876D. This mass corresponds with 5′CCCCCCCCCCCCA3′, to which 4 more C-bases have been added. No intermediate products derived from extension of 5′CCCCCCCCCCCCA3′ by 1, 2 or 3 nucleotides were detected. This suggests that the rate-limiting step of the reaction is associated with template formation by the enzyme, rather than with addition of nucleotides to the primer. As seen in FIG. 9 (red curve), 10 min incubation of 5 μM oligonucleotide with 10 μg/ml Klenow exo⁻ at 37° C. results in conversion of approximately 56% of the oligonucleotide to product. Based on these data, a turnover number of 5.6 min⁻¹ was calculated for the enzyme in reaction to the oligonucleotide extension. Experiments similar to those described above, were performed on 5′GGTGGGGGGGGGA3′ (SEQ ID NO: 17), 5′GGGTGGGGGGGGA3′ (SEQ ID NO: 19), and on 5′GGGGGTGGGGGGA3′ (SEQ ID NO: 21) annealed with 5′CCCCCCCCCCCCA3′. Products of all the above template-primer extensions were analyzed by HPLC and Mass spectrometry as described in FIG. 9. Data of the analysis (Table 3) show that the amount of bases added to primer is equal to the amount of G bases separating T from the 5′-end in the template strand. Turnover of the enzyme in the reaction of strand extension drops with the number of G nucleotides separating T from the 5′-end. This proves that an overall rate of extension is controlled by the rate of template-primer formation, and depends on a number of base pairs perturbed during the template formation. The dependence is neither linear nor exponential; the turnover number is reduced smoothly with an increasing number of bases in the range from 1 to 3; further increase in the number results in sharp reduction of the extension rate (see Table 3).

Conclusion

HPLC and the electrophoretic analysis on commercial preparations of poly(dG)-poly(dC) suggest that they are composed of long C-homopolymers and shorter G-fragments not covalently connected one to another. Overhangs at the ends of poly(dG)-poly(dC) can thus exist as a result of improper matching of the G- and C-homopolymers. Formation of high molecular weight aggregates in solution of the commercial polymer might be the result of overhangs-assisted interaction between the DNA molecules.

A method of poly(dG)-poly(dC) synthesis described here yields uniform polymers, which lack the above disadvantages. The synthesized polymer moves as a single band on electrophoresis (FIG. 1, lane 4), comprises equal amounts of dG and dC nucleotides (FIGS. 5A-B and Table 1), and is composed of dG- and dC-homopolymers having equal lengths (FIG. 2, solid curve). The polymer is efficiently digested by DNase, and is stained with ethidium bromide. The polymer, in contrast to that obtained from Sigma, can thus be considered as a double stranded poly(dG)-poly(dC) comprising two G- and C-homopolymer strands of equal length. The method enables production of poly(dG)-poly(dC) of well-defined length and narrow size distribution of the molecules; polymers varying in size from tens to ten thousand base pairs can be manufactured.

As demonstrated in FIGS. 6A-C and 7, the distance between 5′-ends of double-labeled Flu-(dG)₁₂-(dC)₁₂-TAMRA oligonucleotide (fluorescence of Flu) increases during synthesis suggesting that the labeled 5′-ends of template-primer move in opposite directions during the extension process, leading to formation of complete double stranded poly(dG)-poly(dC) with Flu and TAMRA on its 5′-ends.

A molecular mechanism of strands slippage during the synthesis is not well established. To explain slippage, two models can be considered. In one scenario, one of the strands composing poly(dG)-poly(dC) slides on the other, providing template regions on both the 3′-ends of the polymer which, when filled in by the polymerase, increased the strand's length. A number of successive slippage and replication cycles then leads to a long double-stranded polymer. Sliding of the strand should thus involve complete breakage and reformation of all G-C base pairs of the entire polymer. The activation energy of the process of this reaction is proportional to a number of bases composing poly(dG)-poly(dC) and, if the proceedings are in accordance with the above scenario, the rate of the polymer growth should drop exponentially with a number of bases composing the polymer. The present experiments, however, show that the rate of the synthesis is largely independent of the length of the DNA-fragments being synthesized (FIG. 3). In the second scenario, the enzyme binds to the 3′-end of DNA, shifts the end-nucleotide on the 3′-end of the polymer in 5′-direction and generates a short, single-stranded template and a loop de novo. Formation of a loop is driven by interaction of DNA with polymerase and is associated with melting and rearrangement of hydrogen bonds at the end of poly(dG)-poly(dC). Loop migration through the DNA results in formation of a template region on its opposite end; filling the template by polymerase finalizes a single extension cycle. Loop formation requires pairing of nucleotide at the 3′-end of the primer strand with a complimentary nucleotide in sequence of template strand in accord to the base-pairing rules. In the case of poly(dG)-poly(dC), a nucleotide to which the 3′-end one is paired is located next to the 5′-end one in the sequence. The present inventors have shown that an elementary step of poly(dG)-poly(dC) extension includes addition of one base to each strand of the polymer, thus proving the above suggestion. If proper pairing of the 3′-end nucleotide is not present the synthesis does not take place. The present inventors showed that if the 3′-end nucleotides in poly(dG) and poly(dC) strands were substituted for A one, the polymer did not grow (Table 2). Introduction of T-nucleotide into the complementary strand to allow pairing with the A-nucleotide resulted in restoration of the synthesis. Analysis of the products of 5′CCCCCCCCCCCCA3′-5′GGGGTGGGGGGGA3′ replication by Klenow exo⁻ in the presence of dGTP and dCTP showed, that four C-nucleotides were added to the 5′CCCCCCCCCCCCA3′- strand; 5′GGGGTGGGGGGGA3′-strand was left non-expanded. These data can be explained (FIG. 10) by the assumption that the enzyme binds the oligonucleotide and shifts the A-base at the 3′-end of 5′CCCCCCCCCCCCA3′ until it is becoming paired with the T-nucleotide of 5′GGGGTGGGGGGGA3′-template strand. A single stranded template and a loop de novo are then formed. The template is subsequently filled by polymerase to complete the extension cycle. An overall rate of strand extension is controlled by the rate of template-primer formation. Once formed, a template is rapidly filled by Polymerase before the enzyme-DNA complex dissociates. This conclusion is supported by the following experimental observations: 1—no intermediate products were observed with a number of nucleotides added to 5′CCCCCCCCCCCCA3′, which is less than that separating T-nucleotide from the 5′-end of the complementary strand ; 2—the rate of 5′CCCCCCCCCCCCA3′ extension decreases with an increase of the distance separating T-nucleotide from the 5′-end of the template. The oligonucleotide dissociates from the complex with the enzyme after the template has been filled and a loop relaxes into a structure with an overhang at the 5′-end. The overhang cannot be used as a template for Klenow exo⁻. The later is due to the absence of T-nucleotide in the sequence of template with which A-nucleotide at the 3′-end of the primer could be paired; proper pairing of the 3′-end of the primer strand seems to be strictly required for initiation of the synthesis. The rate of extension depends on the number of bases composing the proposed loop. The inventors have shown that the rate of extension is relatively high for a number of bases between one and three. Only a slight decrease of the extension rate with an increasing number of bases was observed (see Table 3). A further increase in the number of bases resulted in sharp reduction of the extension rate. This is probably due to the high activation energy of the template formation, which strongly limits the rate of extension if the number of bases exceeded three.

The rate of polymer growth is independent of the length of the fragments being synthesized. It has been shown that poly(dG)-poly(dC) as long as 10 Kbase pairs continues to grow at the rate equal to 50 base pairs per minute. If loop migration through the DNA takes place while it is being elongated, the loop should skip through 3 microns (length of extended 10 Kbase pairs DNA) distance in seconds. Movement of a loop over long molecular distances most probably proceeds through a sequence of elementary transfer steps, each including movement of a loop one base pair towards an opposite end of the polymer. This movement includes opening of a G-C pair and thus is determined by base pairs opening dynamics. In general, G-C base pair lifetimes have been found to be approximately equal to 10-20 ms however, in tracts G-C have unusually rapid base pair dynamics, leading to a much higher base pair dissociation constant. Fast rate of poly(dG)-poly(dC) replication can alternatively be explained by the assumption that multiple loops migrate simultaneously in opposite directions through DNA. Such loops can in principle be structurally accommodated in a DNA helix.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A homogeneous population of fully double stranded nucleic acid molecules having blunt ends, each of said molecules in said population being at least 100 base pairs long, each of said molecules in said population having the same repetitive core sequence selected from the group consisting of mono-, di-, or tri-nucleotide, with the proviso that said repetitive core sequence is not A mononucleotide.

2. A homogeneous population of fully double stranded nucleic acid molecules having blunt ends, each of said molecules in said population being at least 1000 base pairs long, each of said molecules in said population having the same repetitive core sequence of an A mononucleotide.

3. The homogeneous population of claim 1, wherein each of said fully double stranded nucleic acid molecules is less than ten kilobases.

4. The homogeneous population of claim 1, wherein said mononucleotide comprises guanine.

5. The homogeneous population of claim 1, wherein a 5′ end of at least one of said fully double stranded nucleic acid molecules is attached to a functional moiety.

6. The homogeneous population of claim 5, wherein said functional moiety is selected from the group consisting of a thiol molecule, a disulfide molecule and a biotinylated molecule.

7. The homogeneous population of claim 1, wherein said mono-, di, or trinucleotide comprises at least one modified base.

8. The homogeneous population of claim 1, wherein at least one of said fully double stranded nucleic acid molecules is immobilized to a solid support.

9. The homogeneous population of claim 1, wherein at least one of said fully double stranded nucleic acid molecules comprises a material selected from the group consisting of a conducting material, a semiconducting material, a thermoelectric material, a magnetic material, a light-emitting material, a biomineral and a polymer.

10. The homogeneous population of claim 9, wherein said conducting material is a transition metal.

11. The homogeneous population of claim 10, wherein said transition metal is selected from the group consisting of silver, gold, copper, platinum, nickel and palladium.

12. The homogeneous population of claim 9, wherein said semiconducting material is selected from the group consisting of a group IV semiconducting material, a group II-VI semiconducting material and a group III-V semiconducting material.

13. The homogeneous population of claim 9, wherein said magnetic material is a paramagnetic material.

14. The homogeneous population of claim 13, wherein said paramagnetic material is selected from the group consisting of aluminum, copper, and platinum.

15. The homogeneous population of claim 9, wherein said magnetic material is a ferromagnetic material.

16. The homogeneous population of claim 15, wherein said ferromagnetic material is selected from the group consisting of magnetite, cobalt, nickel and iron.

17. The homogeneous population of claim 9, wherein said light-emitting material is selected from the group consisting of dysprosium, europium, terbium, ruthenium, thulium, neodymium, erbium, ytterbium and any organic complex thereof.

18. The homogeneous population of claim 9, wherein said biomineral comprises calcium carbonate.

19. The homogeneous population of claim 9, wherein said polymer is selected from the group consisting of polyethylene, polystyrene and polyvinyl chloride.

20. The homogeneous population of claim 9, wherein said thermoelectric material is selected from the group consisting of bismuth telluride, bismuth selenide, bismuth antimony telluride and bismuth selenium telluride.

21. The homogeneous population of claim 5, wherein said functional moiety is a detectable moiety.

22. A method of synthesizing a homogeneous population of fully double stranded nucleic acid molecules having blunt ends, the method comprising reacting a fully double stranded initiator nucleic acid molecule having blunt ends and further having a repetitive core sequence selected from the group consisting of mono-, di-, or tri-nucleotide with nucleotide tri phosphates in the presence of a nucleic acid polymerase, thereby de novo enzymatically synthesizing the homogeneous population of fully double stranded nucleic acid molecules having blunt ends.

23. The method of claim 22, wherein said fully double stranded initiator nucleic acid molecule are at least ten nucleotides long.

24. The method of claim 22, wherein said mono nucleotide comprises guanine.

25. The method of claim 24, wherein said fully double stranded initiator nucleic acid molecule is between ten and twenty.

26. The method of claim 22, wherein said reacting is effected at a non denaturing temperature.

27. The method of claim 26, wherein said non-denaturing temperature is between 25° C. and 37° C.

28. The method of claim 22, wherein said nucleic acid polymerase is exonuclease free Klenow.

29. The method of claim 22, further comprising isolating the homogeneous population of nucleic acid molecules following said reacting.

30. The method of claim 22, wherein said fully double stranded initiator nucleic acid molecule are purified.

31. A kit for de novo synthesizing a homogeneous population of nucleic acid molecules, the kit comprising, in a single container nucleotides and fully double stranded initiator nucleic acid molecules, said fully double stranded initiator nucleic acid molecules having blunt ends and further having a repetitive core sequence selected from the group consisting of mono-, di-, or tri-nucleotide.

32. The kit of claim 31, further comprising polymerase in a separate container.

33. A wire composed of the fully double stranded nucleic acid molecules having blunt ends of claim 1.

34. A fiber composed of the fully double stranded nucleic acid molecules having blunt ends of claim 1.

35. A fabric composed of the fully double stranded nucleic acid molecules having blunt ends of claim 1.

36. An electronic circuit comprising a support and the fully double stranded nucleic acid molecules having blunt ends of claim 1 connected to each other and attached to said support.

37. A composition comprising the homogeneous population of fully double stranded nucleic acid molecules having blunt ends of claim 1.
